(19) United States
(12) Patent Application Publication
(10) Pub. No.: US 2025/0072747 A1
(43) Pub. Date: Mar. 6, 2025

(54) DEVICE AND METHOD FOR MEASURING OCULAR TISSUE CHARACTERISTICS

(71) Applicant: Alcon Inc., Fribourg (CH)

(72) Inventors: Gangjun LIU, Portland, OR (US); Lingfeng YU, Rancho Santa Margarita, CA (US)

(21) Appl. No.: 18/805,430

(22) Filed: Aug. 14, 2024

Related U.S. Application Data
(60) Provisional application No. 63/579,430, filed on Aug. 29, 2023.

(51) Int. Cl. A61B 3/10

(52) U.S. Cl. CPC A61B 3/102

(57) ABSTRACT

An optical coherence tomography (OCT) scanning system for measuring ocular tissue characteristics of a patient's eye. The system includes an OCT scanner configured to implement a scanning sequence on the ocular tissue that scans the ocular tissue with an optical signal to obtain OCT data based on a reflected version of the optical signal. The system includes a computer with a processor configured to provide control signals to the OCT scanner to implement the scanning sequence, and to determine a rate of change of the phase or amplitude data associated with the scans. The system determines a growth constant of the reflected version of the optical signal based on the rate of change of the phase or amplitude data, and determines, based on the growth constant, a diffusion coefficient associated with the cornea. The system determines, based on the diffusion coefficient, a quantitative parameter of the cornea.

### CROSS-REFERENCE TO RELATED APPLICATIONS

This application claim benefit of and priority to U.S. Provisional Patent Application No. 63/579,430, filed Aug. 29, 2023, which is hereby assigned to the assignee hereof and hereby expressly incorporated by reference in its entirety as if fully set forth below and for all applicable purposes.

### INTRODUCTION

Utilizing optical coherence tomography (OCT) scans for observing ocular tissue is known.

### SUMMARY

Some embodiments herein relate to an OCT scanning system for measuring tissue characteristics of a patient's eye. The system includes an OCT scanner configured to implement a scanning sequence via an optical signal on the cornea, lens, iris, conjunctiva, retina and other ocular tissue. The scanning sequence includes performing a plurality of scans of the eye to obtain OCT data. OCT data is based on a reflected version of the optical signal; the OCT data being representative of phase, intensity/amplitude or both phase and amplitude data of the reflected version of the optical signal. In some embodiments, the system includes a computer including a processor.

In some embodiments, the processor is configured to provide control signals to the OCT scanner to implement the scanning sequence and determine a rate of change of the signal data associated with the plurality of scans. The rate of change of the signal data characterizes a change of the reflected version of the optical signal as a function of time. In some embodiments, the processor determines a growth constant of the reflected version of the optical signal based on the rate of change of the phase data and determines, based on the growth constant, a diffusion coefficient associated with the cornea. In some embodiments, the processor determines, based on the diffusion coefficient associated with the tissue, a quantitative parameter of the ocular tissue.

Some embodiments include a method of measuring ocular tissue characteristics, such as ocular thickness, topography and curvature. The method includes the use of an optical coherence tomography (OCT) device and a computer coupled to the OCT device. The method may be performed by a computer in communication with an OCT device. Some embodiments include performing a plurality of scans of the ocular tissue to obtain OCT data representative of an OCT signal including phase and amplitude information measuring ocular tissue characteristics in real-time at multiple ocular locations.

In some embodiments, the method includes, at the OCT device, determining a rate of change of amplitude data or phase data or the data combining both amplitude and phase data associated with the plurality of scans, and the rate of change of the data characterizing a change of the reflected version of the optical signal as a function of time. In some embodiments, the method includes determining a growth constant of the reflected version of the optical signal based on the rate of change of the data. In some embodiments, the method includes determining, based on the growth constant, a diffusion coefficient associated with the ocular tissue. In some embodiments, the method includes determining, based on the diffusion coefficient associated with the ocular tissue, a quantitative parameter of the ocular tissue.

Some embodiments include a computer-program product, comprising a non-transitory computer-readable medium. Some embodiments include a computer-program product comprising a non-transitory computer-usable medium having computer-readable program code embodied therein. In some embodiments, the computer-readable program code is adapted to be executed by one or more processors to implement a method. In some embodiments, the method comprises receiving, from an OCT device, OCT data representative of an OCT signal including phase and amplitude information, the OCT data being generated by performing a plurality of scans of ocular tissue.

In some embodiments, the method includes determining a rate of change of amplitude data or phase data associated with the plurality of scans, the rate of change of the phase data or the amplitude data characterizing a change of the reflected version of the optical signal as a function of time. In some embodiments, the method includes determining a growth constant of the reflected version of the optical signal based on the rate of change of the amplitude data or the phase data. In some embodiments, the method includes determining, based on the growth constant, a diffusion coefficient associated with the ocular tissue. In some embodiments, the method includes determining, based on the diffusion coefficient associated with the ocular tissue, a quantitative parameter of the ocular tissue.

### BRIEF DESCRIPTION OF THE DRAWINGS

So that the manner in which the above-recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, and may admit to other equally effective embodiments.

FIG. 1 illustrates an example configuration of an ophthalmic diagnostics system, in accordance with one or more embodiments.

FIG. 2 is a block diagram of various components of the ophthalmic diagnostics system of FIG. 1, in accordance with one or more embodiments.

FIG. 3 illustrates exemplary aspects of an ophthalmic diagnostics system, in accordance with one or more embodiments.

FIGS. 4A-4D illustrate exemplary processes for measuring ocular tissue characteristics, in accordance with one or more embodiments.

FIGS. 5A-5B illustrate exemplary processes for measuring ocular tissue characteristics, in accordance with one or more embodiments.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

The present disclosure will now be described in detail with reference to the drawings, which are provided as illustrative examples of the disclosure so as to enable those skilled in the art to practice the disclosure. Notably, the figures and examples below are not meant to limit the scope of the present disclosure to a single embodiment, but other embodiments are possible by way of interchange of some or all of the described or illustrated elements. Moreover, where certain elements of the present disclosure can be partially or fully implemented using known components, only those portions of such known components that are necessary for an understanding of the present disclosure will be described, and detailed descriptions of other portions of such known components will be omitted so as not to obscure the disclosure.

As used herein, the singular form of “a,” “an,” and “the” include plural references unless the context clearly dictates otherwise. As used herein, the statement that two or more parts or components are “coupled” shall mean that the parts are joined or operate together either directly or indirectly (i.e., through one or more intermediate parts or components, so long as a link occurs). As used herein, “directly coupled” means that two elements are directly in contact with each other. As used herein, “fixedly coupled” or “fixed” means that two components are coupled so as to move as one while maintaining a constant orientation relative to each other. As used herein, “operatively coupled” means that two elements are coupled in such a way that the two elements function together. It is to be understood that two elements “operatively coupled” does not require a direct connection or a permanent connection between them. As utilized herein, “substantially” means that any difference is negligible, such that any difference is within an operating tolerance that is known to persons of ordinary skill in the art and provides for the desired performance and outcomes as described in the embodiments described herein. Descriptions of numerical ranges are endpoints inclusive.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality). Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

In the exemplary embodiments described herein, an embodiment showing a singular component should not be considered limiting; rather, the disclosure is intended to encompass other embodiments including a plurality of the same component, and vice-versa, unless explicitly stated otherwise herein. Moreover, applicants do not intend for any term in the specification or claims to be ascribed an uncommon or special meaning unless explicitly set forth as such. Further, the present disclosure encompasses present and future known equivalents to the known components referred to herein by way of illustration.

The embodiments described herein provide a system and methods for measuring ocular tissue characteristics including, ocular thickness, stiffness, topography and curvature. In some embodiments, an optical coherence tomography (OCT) scanning system is configured for evaluating such ocular tissue characteristics. Functional extension of OCT is advantageous for early diagnosis of diseases by providing psychological information beyond morphological structure, without injecting contrast agent. For example, one functional extension of OCT is to detect motion effects such as blood flow, tissue movement, Brownian motion, and/or dynamic scattering by utilizing phase correlation of various components of an OCT signal, which is discussed in further detail below.

Some phase decorrelation techniques, however, require a specialized type of OCT system (i.e., PhD OCT), which use only one single beam system with a specific scanning pattern (the M-B mode scan). Such techniques may be problematic, however, because, during in-vivo human testing, the M-B scan pattern used by the PhD OCT will require a few seconds to capture a B-scan image. Even with eye tracking tools, both the structure and the growth value are distorted by inherent eye motion of the patient. A correction must be implemented for such distortion.

Thus, some embodiments described herein determine physiological information of the ocular tissue by analyzing the intensity and/or phase of an OCT signal using an OCT system without further specialization. In some embodiments, tissue property parameters of the ocular tissue may be extracted from either intensity or phase of an optical signal. In some embodiments, multiple measurements taken at various locations and different times may be compared and averaged to provide more accurate results, which is described in further detail below. Advantageously, motion effects may be detected by implementing at least two OCT signals, which are acquired at the same location (or substantially the same location). Such multiple signals may be compared and analyzed to ascertain the motion effects in the cornea and/or corneal tissue characteristics such as blood flow, tissue movement, Brownian motion, and/or dynamic scattering of light caused by ocular tissue particles, which is discussed in detail below.

In some embodiments, parallel multiple beams may be used to extract the ocular tissue characteristics simultaneously at multiple locations. Simultaneous multiple location measuring proves faster and more stable measurement with minimum distortion, when compared to measuring in the same location at different time. In some embodiments, a B-M scan pattern may be used to provide ocular image without distortion. In some embodiments, multiple B-scan images acquired at the same location could be used to create high-definition ocular images, which is discussed in detail below. Implementing the embodiments described herein, provides improved OCT scanning while avoiding injection of contrast agent and removing any requirement to modify conventional OCT systems (e.g., PhD OCT).

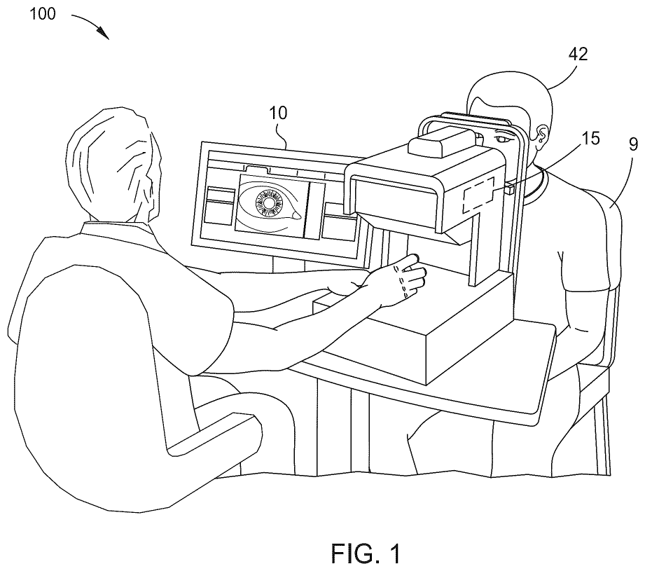

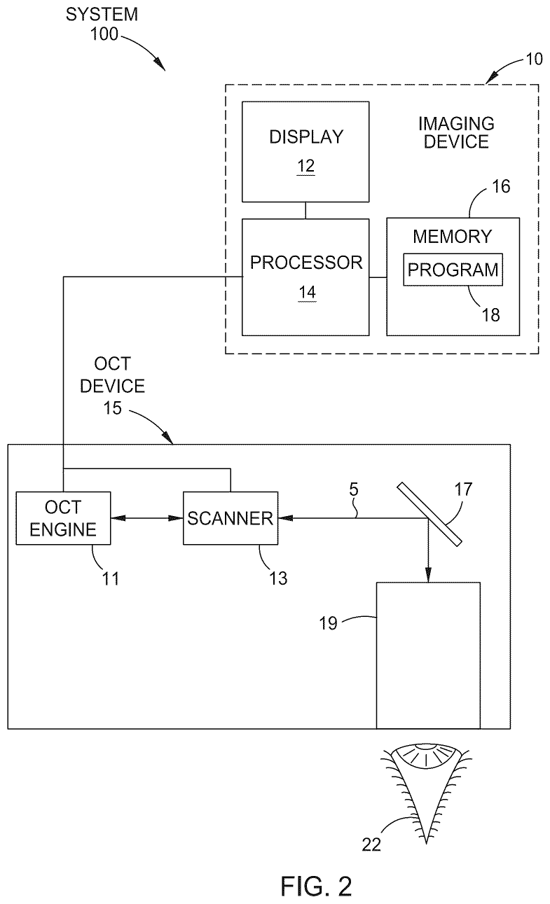

Referring now to FIGS. 1-2, FIG. 1 illustrates an example of an ophthalmic diagnostics system 100 (hereinafter “system 100”) according to some embodiments. System 100 may be used for different types of diagnostic and treatment procedures. For example, system 100 may be used for diagnosis of glaucoma, keratoconus, and/or ectasia. In addition, or alternatively, system 100 may be used to provide data for personalizing LASIK or cataract surgery. As shown in FIG. 1, system 100 may include a control/processing unit 10 and an OCT device 15; patient 42 sits in a chair 9.

As shown in FIG. 2, in some embodiments, system 100 includes OCT device 15 in communication with control/processing unit 10, coupled as shown. OCT device 15 includes controllable components, such as OCT engine 11, scanner 13, one or more optical elements/dichroic mirror 17, and/or focusing objective 19, coupled as shown. Control/processing unit 10 includes display 12, processor 14, and memory 16, coupled as shown. Memory 16 stores a computer program 18 and data.

In some embodiments, control/processing unit 10 may control components of system 100 in accordance with computer program 18. For example, control/processing unit 10 controls components (e.g., OCT engine 11, scanner 13, to focus the imaging light beam of OCT engine 11 at a desired location on eye 22, such as a desired location on an ocular surface thereof. In some embodiments, memory 16 may store information used by control/processing unit 10. For example, memory 16 may store images of eye 22, OCT data, and/or other suitable information, and control/processing unit 10 may access information from memory 16.

In various embodiments, the computer program 18 includes functionality, such as controlling the scanning of the imaging light beam, which may be directed by a user such as a medical professional. In some embodiments, control/processing unit 10 can measure ocular tissue characteristics of eye 22, which is discussed in further detail below. Some embodiments include a computer-program product, comprising a non-transitory computer-readable medium. Some embodiments include a computer-program product comprising a non-transitory computer-usable medium having computer-readable program code embodied therein. In some embodiments, the computer-readable program code is adapted to be executed by one or more processors to implement the methods described herein.

With particular reference to OCT device 15, OCT engine 11 generates and emits an optical signal 5. Optical signal 5 may be guided to tissue of an eye 22 of patient 42. For example, optical signal 5 may be guided to an ocular surface of the eye 22. Scanner 13 laterally ally directs optical signal 5. The lateral direction refers to directions orthogonal to the direction of beam propagation. Scanner 13 may laterally direct in any suitable manner. For example, scanner 13 may include a pair of galvanometrically mirrors that can be tilted about mutually perpendicular axes. As another example, scanner 13 may include an electro-optical crystal that can electro-optically steer optical signal 5.

One or more optical elements 17 direct the imaging light beam towards focusing objective 19. Optical element 17 may act on (e.g., transmit, reflect, refract, diffract, collimate, condition, shape, focus, modulate, and/or otherwise act on) the imaging light beam. In some embodiments, optical elements may include one or more of: a lens, prism, mirror, diffractive optical element (DOE), holographic optical element (HOE), and/or spatial light modulator (SLM). In some embodiments, optical element 17 may include a mirror. In some embodiments, optical element 17 may include a dichromic mirror. Focusing objective 19 focuses the imaging light beam towards a portion of the eye 22, such as the ocular surface of eye 22. In some embodiments, focusing objective 19 may include an objective lens (e.g., an f-theta objective).

OCT engine 11 receives a returned imaging light beam contained in optical signal 5. The propagating portion of optical signal 5 reflects off the surface of ocular tissue via backscattering from the eye 22, along the opposite direction of the imaging light beam. Backscattering occurs when light beams deflect, refract and/or diffract through the structures of ocular tissue in eye 22. In some embodiments, OCT engine 11 may be configured to generate an image (via display 12) or images to provide actionable feedback for storage, as described below in detail.

For example, in various embodiments, OCT engine 11 is configured to interferometrically analyze the returned imaging light beam to provide OCT data representing a position-dependent structural property of the eye 22, such as structural property of a cornea thereof. For example, OCT engine 11 may be configured to provide OCT data representing an image of the cornea/lens/iris/retina at or in the vicinity of the focal position x,y,z and to provide OCT data representing a position-dependent optical density n (x,y,z) of the ocular tissue as well as a position-dependent mass density p (x,y,z) of the ocular tissue.

Although certain examples of OCT device 15 are described above, in various embodiments, OCT device 15 may be configured to conduct different types of OCT scans, which is described in further detail below. In some embodiments, OCT device 15 may be configured to execute an M-scan. In other embodiments, OCT device 15 may be configured to execute a B-scan. In yet other embodiments, OCT device 15 may be configured to execute an MB-scan. In some embodiments, OCT device 15 may be configured to execute a BM-scan, which is discussed in further detail below. Other types of OCT scans will be apparent to one skilled in the art after a detailed review of the present disclosure.

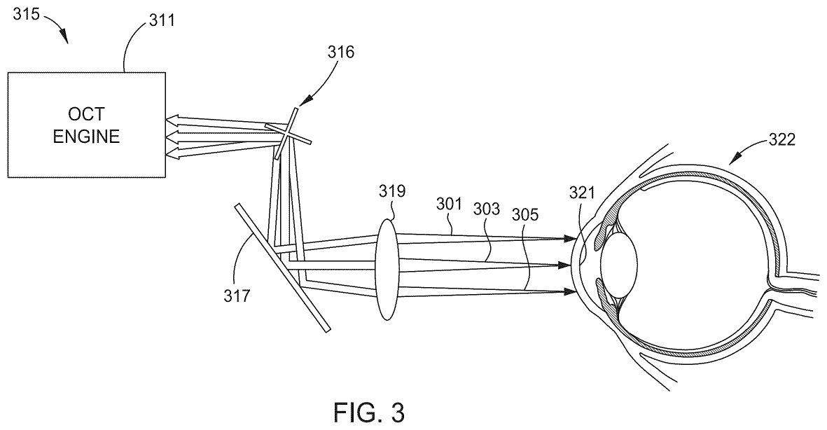

Referring now to FIG. 3 in conjunction with FIGS. 1-2, FIG. 3 illustrates an example of OCT device 315. In general, the OCT device 315 may include any of the components and functionality described relative to OCT device 15 of FIGS. 1-2. Similarly, OCT device 15 may include any of the components and functionality described relative to OCT device 315 Therefore, for ease of description, like components of system 100 and OCT device 315 may be periodically referenced interchangeably. For simplicity, the illustration of FIG. 3 focuses on an example OCT device 315.

OCT device 315 includes OCT engine 311, scanner 313, optical elements 317, and focusing objective 319. In general, the OCT engine 311, scanner 313, optical elements 317, and the focusing objective 319 each operate as described relative to OCT engine 11, scanner 13, optical elements 17, and/or the focusing objective 19, respectively, of FIG. 2. In the example of FIG. 3, the one or more optical elements 317 are shown as a dichroic mirror, focusing objective 19 is shown as an objective lens.

In similar fashion to OCT engine 11 of FIG. 2, OCT engine 311 generates and emits an optical signal 301 that propagates an imaging light beam that is guided to, or propagates onto, a surface of a ocular tissue 321 of an eye 322, and may thereafter receive a returned imaging light beam, backscattered from the eye 322, along the opposite direction of the imaging light beam. In some embodiments, OCT engine 311 emits multiple optical signals for example three optical signals 301, 303, 305. In some embodiments, OCT engine 311 may utilize two optical signals, which is discussed in further detail below.

As shown in FIG. 3, some embodiments herein advantageously implement multiple, parallel beams (e.g., 301, 303, 305), which may be used for extracting physiological information of ocular tissue 321, simultaneously at multiple locations, which facilitates faster and more stable measurement with minimum distortion when compared to a taking multiple measurements at a single location with a single beam, which is discussed in detail further below.

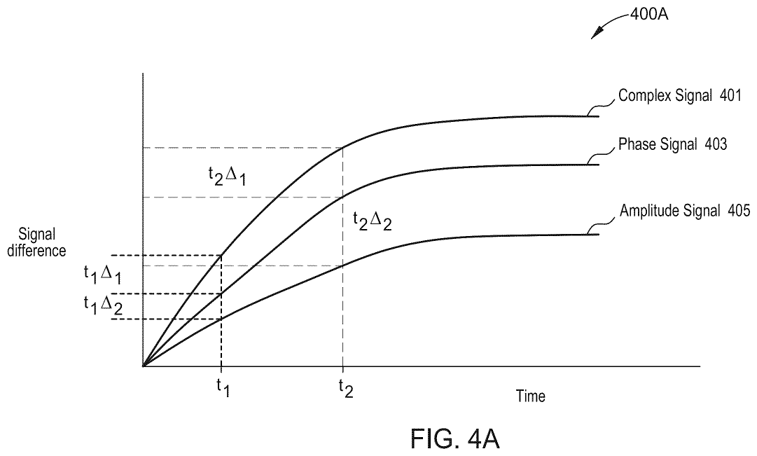

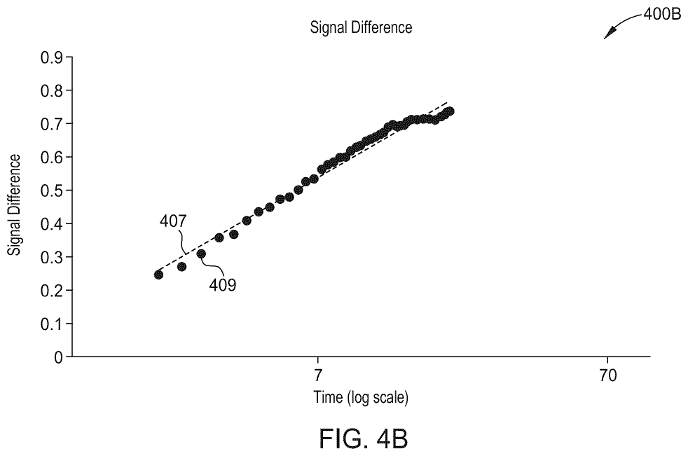

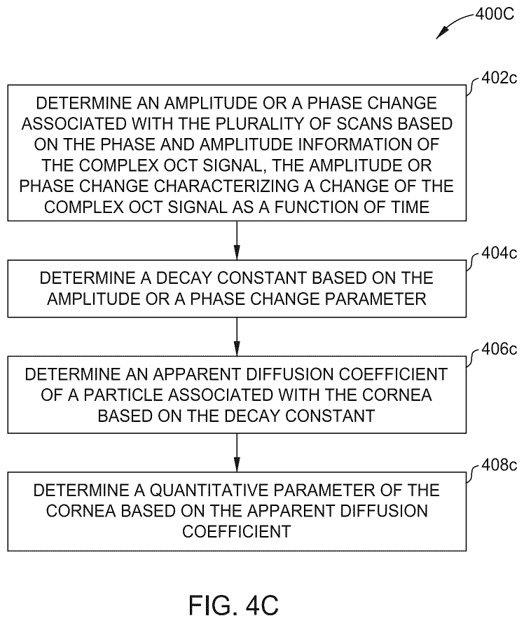

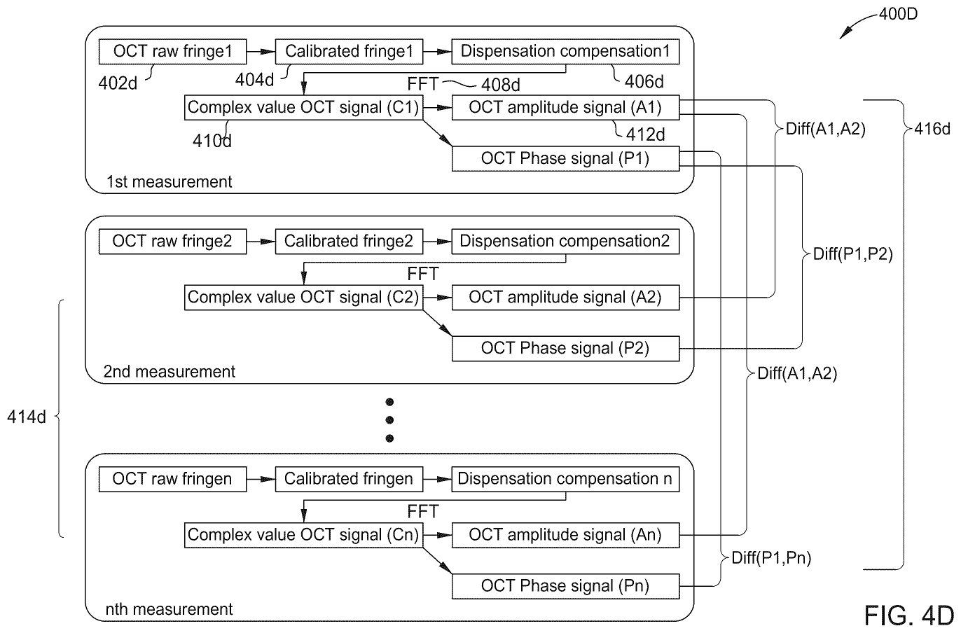

Referring now to FIGS. 4A-4C, in conjunction with FIGS. 1-3, FIG. 4A depicts a graph 400A depicting a relationship between time and signal difference of components of an optical signal (e.g., 301, 303, 305) emitted by OCT engine 311. Optical signal components includes a complex signal 401, phase signal 403, or amplitude signal 405.

Discussed in further detail below, in some embodiments, a difference (e.g., t₂Δ₂-t₁Δ₁) between the amplitude (Intensity), phase or the complex signals are used to obtain the motion signals inside ocular tissue 321. In order to quantify hard to detect motion inside ocular tissue 321, some embodiments implement phase-decorrelation OCT to measure the growth constant of the time-dependent complex-value change. The growth constant value may be utilized for quantifying ocular tissue 321 characteristics, which is discussed further below.

For clarity, in OCT systems (e.g., 100), detected signals are often interference fringes. By performing a Fourier transform on interference fringes to convert said signal from the optical domain (wavelength or time) to the depth domain provides depth-resolved OCT signals. The depth-resolved OCT signals are represented as complex values, which contain both amplitude (intensity) and phase information. The complex value signal can be represented as:

$$E = I * {e}^{( - i Φ )} \qquad Eqn . ( 1 )$$

In Eqn. 1, E is the complex signal, I is the amplitude (where intensity is square of I) of the signal, Φ is the phase of the signal, and i is the imaginary unit (i²=−1). The exponential term e^{(-1Φ)} is a compact way to represent the phase information, combining both the real and imaginary components of the complex signal in one expression.

The complex value signal (E) is the result of the interference pattern (i.e., fringe(s)) created when the reference (i.e., propagating portion of optical signal 5) and sample beams (i.e., reflected portion of optical signal 5) recombine in the OCT system (e.g., 100). The amplitude (I) represents the strength of the reflected light, while the phase (Φ) carries information about the relative path length difference between the reference and sample beams.

Thus, the amplitude and phase information contained in the complex signal may be used to generate high-resolution, cross-sectional images of the tissue and may advantageously provide insights into ocular tissue's optical and biomechanical characteristics such as stiffness, which is discussed further below.

As shown in FIG. 4A, in OCT device 315, the signal difference between multiple acquired OCT signals reflected from the same location sequentially may increase with the increase of the time difference between the acquired signals. The signal difference increase over time, may be utilized for quantifying the motion (such as flow or Brownian motion) strength corresponding to ocular tissue 321. The relationship between signal differences between acquired OCT signals at the same location taken at different times may be fitted with an exponential growth function. For example, as shown in FIG. 4B, the solid circles plot measured signal difference 407, while the dashed line represents an exemplary exponential growth function fitted curve 409.

In FIG. 4B, discussed in detail below, the growth constant of the phase-decorrelation and time relationship is directly related to the Brownian motion diffusion coefficient and may be used to quantify ocular tissue 321 characteristics. For example, by comparison of the measured Brownian diffusion coefficient to clinical test data. For clarity a brief review follows:

In OCT, phase-decorrelation signal processing may be utilized for measuring the motion of scattered photons in tissue, which is often caused “scatterers” within ocular tissue 321. Discussed in detail below, “scatterers” generally refer to particles or interfaces within ocular tissue 321 that cause light to scatter.

When OCT signal (e.g., 5, 301, 303, 305) is incident onto ocular tissue 321, light from such OCT signal penetrates ocular tissue 321 to a depth depending on ocular tissue 321 characteristics, discussed further below. As light from the OCT signal travels through ocular tissue 321, such light encounters different ocular structures, such as cells, organelles, collagen fibers, ocular layers, and other known microscopic structures. Each of these structures has different optical properties, such as refractive index. When light from OCT signal encounters such structures, some light is reflected back, and some light is scattered, (i.e., the light is deflected off in various directions rather than going straight through). Such structures inside ocular tissue 321 that cause the scattering of light are herein referred to as “scatterers”.

Analysis of the scattering of light may be advantageous as such scattered light may be collected and measured by OCT device 315 to form an image. For example, discussed in further detail below, by measuring the time delay and intensity of the returned light in optical signal 301, 303, 305, OCT device 315 is capable of constructing a depth-resolved image of ocular tissue 321. The distribution, size, and nature of the scatterers within ocular tissue 321 contribute to the specific features and contrast seen in OCT images produced by OCT device 315. Such OCT images (i.e., OCT data), may be displayed to facilitate visualizing, over time, the motion of internal microstructures in ocular tissue 321 in a non-invasive manner.

Discussed in further detail below, by visualizing motion of ocular tissue 321 internal microstructures, or particles, within ocular tissue 321, the Brownian motion, or apparent Brownian motion, of such particles may be determined. Brownian motion refers to the random motion of particles suspended in ocular tissue 321 resulting from collisions of such particles with other randomly moving molecules and/or particles within ocular tissue 321.

As utilized herein, “apparent Brownian motion,” refers to OCT device 15 observations where the measured motion of ocular tissue 321 particles appears to be random and diffusive, similar to true Brownian motion, but such motion may also be influenced by other factors including heat, light, and ambient electromagnetic fields. For example, the measured motion by OCT device 315 of scatterers within ocular tissue may be affected by fluid flow within ocular tissue, cardiac or respiratory motion of the patient, and/or instrumental and environmental noise or artifacts. Such factors may contribute to the apparent random motion observed in ocular tissue particles via OCT device 315.

As implemented herein, “apparent Brownian motion” means that motion may appear Brownian, and may also include non-Brownian motion influenced by such other factors. Further, as utilized herein the term “apparent diffusion coefficient” quantifies the magnitude of such apparent random/Brownian motion, which may be analyzed for determining ocular tissue characteristics, microstructure and physiology as discussed herein.

Thus, as discussed above, OCT signal 301, 303, 305 is affected by Brownian motion of scatterers within ocular tissue 321. The motion of such scatterers causes changes in the phase and amplitude of OCT signal 301, 303, 305 over time. By comparing such phase or amplitude of the OCT signal over time from successive OCT scans (e.g., A-lines, discussed further below), the embodiments herein determine the phase or amplitude change between such successive scans (i.e., phase/amplitude decorrelation). Such phase/amplitude decorrelation is advantageous for measuring or quantifying how much the phase changes from one scan to a subsequent scan, which is discussed in further detail below.

In some embodiments, system 100 may model and display a relationship between phase decorrelation and time in OCT signal 301, 303, 305, wherein such modeling may be characterized by a growth function. Such growth function, may advantageously be expressed by a growth constant, which captures the rate of change in phase decorrelation over time.

Accordingly, the embodiments herein provide a manner of connecting the captured rate of change in phase decorrelation to the Brownian motion of scatterers within ocular tissue 321. Because the Brownian motion within ocular tissue 321 causes the phase decorrelation over time, as discussed above, the rate at which the phase decorrelation growths (i.e., the growth constant) may be directly related to the Brownian diffusion coefficient, which quantifies the random motion of the scatterers. The more motion, the faster the phase decorrelation growths, and thus the higher the Brownian diffusion coefficient.

Thus, by measuring the growth constant of the phase-decorrelation and time relationship in OCT signal 301, 303, 305, the embodiments described herein determine the Brownian diffusion coefficient of ocular tissue 321. And such diffusion coefficient may be utilized to quantify characteristics of ocular tissue 321, such as ocular tissue stiffness. For example, in the cornea, because the scatterers are embedded within ocular tissue 321, the motion of such scatterers are affected by the properties of the surrounding ocular tissue, including stiffness. Thus, by determining the diffusion coefficient, system 100 may deduce ocular tissue 321 stiffness. The embodiments above may analogously be applied to amplitude of the OCT signal or to a combination signal of both amplitude and phase.

Thus the embodiments described herein provide a direct measurement which may be correlated with ocular biomechanics and utilized to diagnose, prevent, and treat ocular diseases, which is described in further detail below] For example, techniques involving the evaluation of the water level or water quantity as well as the degree of collagen confinement inside the ocular tissue for determining the ocular biomechanics and diffusion coefficient

FIG. 4C illustrates an example of operations 400C for measuring ocular tissue characteristics. In some embodiments, operations 400C may be implemented by a system that is capable of processing OCT data (e.g., system 100). Although any number of systems, in whole or in part, may implement operations 400C, to simplify discussion, operations 400C will be described in relation to example components of system 100 of FIGS. 1-3.

Thus, based on the relationships between signal differences in acquired OCT signals discussed above, the embodiments described herein implement operations 400C for determining ocular tissue characteristics. Operations 400B may begin at an operation 402c by determining an amplitude or a phase change associated with a plurality of scans based on the phase and amplitude information of the complex OCT signal. The amplitude or phase change characterizes a change of the complex OCT signal as a function of time. Operation 402c may be performed by OCT device 315.

At an operation 404c, OCT device 315 may determine a growth constant based on the amplitude or a phase change parameter. At an operation 406c, OCT device 315 may determine an apparent diffusion coefficient of a particle associated with the ocular tissue based on the growth constant. At an operation 408c, OCT device 315 may determine a quantitative parameter of the ocular tissue based on the apparent diffusion coefficient or the growth constant. The quantitative parameter may be compared to clinical data for ascertaining a condition or disease of the patient's eye

Referring now to FIG. 4D, FIG. 4D describes operations 400D for determining an amplitude or a phase change associated with the amplitude, phase and/or complex signal of an optical signal (e.g., 301, 303, 305) emitted from OCT engine 311 (e.g., operation 402c of FIG. 4C). The amplitude, phase, and/or complex number portion (e.g., 401, 403, 405) of optical signal 301, 303, 305 may be utilized for determining ocular characteristics per operations 400C discussed above. In operations 400D, when determining the signal difference, absolute difference, decorrelation, variance, or other methods may be used.

As implemented herein, a data set may include at least three curves obtained by OCT engine 311 (e.g., phase signal 403). Such curves may be used to determine corresponding growth values for each curve. For example, at least three growth values may be averaged to obtain a more accurate, or optimized, growth value. As shown in FIG. 4D, a total of n times of measurement at the same location may be performed. In some embodiments, the n measurements may be performed sequentially or non-sequentially, utilizing BM mode scanning or MB mode scanning, which is discussed in further detail below.

As shown in FIG. 4D, acquired “fringes” from the measurements are calibrated and dispensation compensation is performed. The term “fringe” in the context of optical coherence tomography (OCT) refers to the interference pattern produced when a reference beam and sample beam recombine after being split by a beam splitter in system 100. Such interference patterns/fringes, contain depth information about ocular tissue 321.

For clarity, in OCT, a low-coherence light source is used to generate a beam of light that is split into two beams: a reference beam and a sample beam. The sample beam is directed towards the tissue being imaged, while the reference beam is directed towards a reference mirror or reflective surface. Both beams are reflected back to the beam splitter and recombine. The recombination of the reference and sample beams creates an interference pattern, or fringe, which depends on the path length difference between the reference and sample beams. The fringe pattern carries information about the depth and reflectivity of the tissue layers, as the light reflected from different depths within the tissue will have different path length differences relative to the reference beam.

In some embodiments, system 100 detects fringe patterns, which are analyzed to extract the depth information. Such depth information may be used to generate high-resolution, cross-sectional images of ocular tissue 321. As discussed above, performing a Fourier transform on the detected fringe signal and converting from the optical domain (wavelength or time) to the depth domain provides the depth-resolved OCT signals. The OCT signals (e.g., 301, 303, 305) are complex numbers, from which/growth values may be derived utilizing known techniques.

Thus, in some embodiments, operations 400D includes performing a first OCT measurement at a location within an eye using system 100. In operation 402d, system 100 determines a first fringe based on the first OCT measurement and calibrates the first fringe in operation 404d. Next, at an operation 406d, OCT device 315 may perform dispersion compensation on the first fringe. In system 100, dispersion compensation corrects for mismatched dispersion properties of the sample and reference signals. Such dispersion mismatch may be caused by different optical paths that the light takes in the sample and reference arms of the interferometer of OCT engine 311. Performing dispersion compensation may include adjusting the optical path lengths or the dispersion properties of the reference signal to match that of the sample signal. By applying dispersion compensation, system 100 may maintain high resolution and contrast in generated images, enhancing the accuracy of the observations and measurements derived from such images

At an operation 408d, OCT device 315 performs a first Fast Fourier Transform (FFT) on the calibrated and compensated first fringe, the output of the FFT may correspond to a data set, as described above. At an operation 410d, OCT device 315 may determine, based on a first output of the FFT, a first depth-encoded OCT signal, wherein the first depth-encoded OCT signal corresponds to a first set of complex numbers. At an operation 412d, system 100 may determine, based on the first set of complex numbers, an amplitude and a phase of the first depth-encoded OCT signal, as discussed above.

At operation 414d, operations 402d-412d are repeated for n measurements. For example, in some embodiments, system 100 may perform a second OCT measurement at the location within the eye using system 100. System 100 then determines a second fringe based on the second OCT measurement and may calibrate the second fringe. Next system 100 performs dispersion compensation on the second fringe and performs a second FFT on the calibrated and compensated second fringe. System 100 may then determine, based on a second output of the FFT, a second depth-encoded OCT signal, wherein the second depth-encoded OCT signals correspond to a second set of complex numbers. Then system 100 determines, based on the second set of complex numbers, an amplitude and a phase of the second depth-encoded OCT signal.

At operation 416d, system 100 determines a signal difference of the first and second OCT measurement based on comparing the amplitude and/or phase shift of the first depth encoded OCT signal to the amplitude or phase shift of the second depth encoded OCT signal. In some embodiments, the first OCT measurement and the second OCT measurement are performed simultaneously in different locations of the cornea. For example, a middle portion, top portion and/or bottom portion of ocular tissue 321. In some embodiments, the different locations may be locations that are spaced apart from one another by at least 0.1 mm.

In order to determine the growth function, system 100 may plot a curve relating the signal differences to the time differences between the acquired OCT signals (e.g., 401, 403, 405). System 100 may fit the plotted curve with an exponential growth function to obtain at least three growth values. Weighted averaging the at least three growth values obtains an optimized growth value, as described above.

System 100 determines, based on the optimized growth value, ocular tissue characteristics, similar to or the same as discussed above, wherein the optimized growth value of a phase-decorrelation and time relationship is directly related to a Brownian diffusion coefficient. In some embodiments determining the signal difference may include utilizing one or more of: absolute difference, decorrelation, and/or variance calculations. Some embodiments described above may further implement: Doppler OCT, Doppler variance OCT, intensity-based Doppler, speckle variance, phase variance, optical micro-angiography, amplitude decorrelation, phase decorrelation, and/or dynamic scattering. In various embodiments, the resultant ocular tissue characteristics may be stored in relation to the patient in the memory 16 or other storage. In addition, or alternatively, the resultant ocular tissue characteristics can be displayed to a user or operator of system 100.

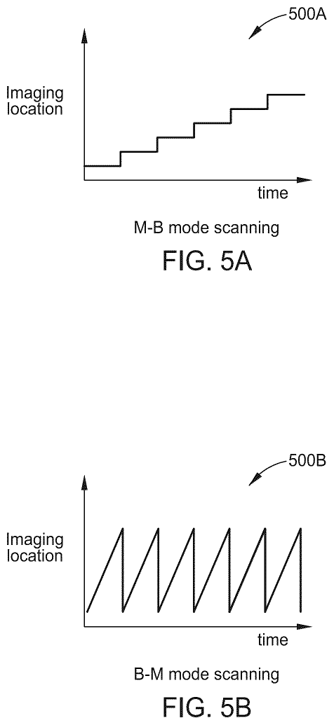

Referring now to FIGS. 5A-5B in conjunction with FIGS. 1-4C, FIG. 5A depicts a graph 500A which depicts the relationship between imaging location and time of a laser beam conducting M-B mode scanning. FIG. 5B depicts a graph 500B showing the relationship between imaging location and time a laser beam conducting B-M mode scanning. A brief overview follows:

OCT systems offer various scanning modes to acquire different types of images or datasets, depending on the clinical requirements and the eye structure being examined. A-scan is a one-dimensional depth profile of a sample or tissue. By measuring the interference patterns produced by the reflected light, system 100 may obtain information about the depth and reflectivity of the tissue layers, creating a depth profile. A-scans may be implemented for measuring the axial length of eye 322, as well as to assess the anterior chamber depth and lens thickness. Such information is advantageous for diagnosing and monitoring conditions of the eye 322. M-scan is multiple A-scans at the same location.

B-scan involves acquiring a single cross-sectional image (B-scan) of the tissue by performing multiple A-scans (i.e., a linear scan along a specified axis). B-scan are scanning modes that provide a two-dimensional view of the tissue's internal structure. In some embodiments, system 100 may acquire multiple B-scans at adjacent locations to create a three-dimensional dataset of ocular tissue 321, known as volume scan or C-scan. Volume scan mode provides a comprehensive view of the tissue structure and is useful for visualizing the entire macular region or analyzing the optic nerve.

As shown in FIG. 5A, M-B mode scan is a term that combines M-mode (multiple A-scan at the same location) and B-mode (at different location) scans. In ophthalmology, M-B mode scans may be implemented in the evaluation of eye 322 and the orbit.

M-B mode scanning combines both M-mode and B-mode techniques, providing a comprehensive assessment of both the static and dynamic aspects of the eye. M scans facilitate extraction the exponential growth curve for each location. B-can allows one to form the cross-sectional image.

As shown by the step-wise progression of graph 500A, M-B mode scanning stays at the same location for a period of time subsequent, then changing locations to a next location and stay for the same amount of time. In M-B scan mode, the M-scans at the same location will allow one to acquire curve about the amplitude/phase/complex signal difference versus time and obtain tissue biomechanics at the specific location. By changing the locations with B-scan, one can know the biomechanics at different locations. In some embodiments, in addition to M-B scan mode, which stays at the same location to acquire multiple A-lines then moves to another location, as discussed above, the B-M scan mode may be utilized.

In some embodiments, when a fast OCT processing is advantageous, the B-M scan mode is advantageously implemented as follows: In some embodiments, B-M mode shown in graph 500B may be used simultaneously or in combination to provide a comprehensive assessment of both the static and dynamic aspects of ocular tissue 321. The number of A-lines in each B-scan of the B-M scan mode will depend on the A-line speed. When a single B-scan can be finished in less than 1 milliseconds, the B-M scan will be preferred.

In some embodiments, the first B-scan image in the B-M scan may be utilized as base-line image to register subsequent B-scan images. The registered B-scan images form a dataset for further processing. In some embodiments, such registered images/data sets may be averaged to get a de-speckled, high-resolution image. The averaged dataset may be used to obtain the growth values or other parameters for the evaluation of ocular characteristics.

Thus the above-described embodiment may provide a diagnostic device with sub-micron resolution that may measure biomechanical properties of diseased or healthy ocular tissue. One or more embodiments described above may be implemented for designing personalized treatment procedure for cataract and LASIK surgery, improving the predictability of treating astigmatism in cataract surgery, identifying patients at risk for complications post-LASIK, and/or improving predictability of ocular treatments. Ascertaining such information with the embodiments described above may facilitate inclusion of new metrics in treatment planning algorithms, which increases predictability of corneal treatments and surgeon confidence. Being able to measure biomechanical properties and using such results in treatment algorithms supports more accurate: cataract outcomes from patient-specific SIA, LRI outcomes from patient-specific calculations, treatment decisions for corneal refractive Px, and Ortho-k outcomes.

The above-disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description.

What is claimed is:

1. An optical coherence tomography (OCT) scanning system for measuring characteristics of ocular tissue of a cornea in a patient's eye, the system comprising:
an OCT scanner configured to implement a scanning sequence via an optical signal on the ocular tissue, the scanning sequence including performing a plurality of scans of the ocular tissue to obtain OCT data based on a reflected version of the optical signal, the OCT data being representative of phase data of the reflected version of the optical signal; and
a computer including a processor configured to:
provide control signals to the OCT scanner to implement the scanning sequence;
determine a rate of change of the phase data associated with the plurality of scans, the rate of change of the phase data characterizing a change of the reflected version of the optical signal as a function of time;
determine a growth constant of the reflected version of the optical signal based on the rate of change of the phase data;
determine, based on the growth constant, a diffusion coefficient associated with the cornea; and
determine, based on the diffusion coefficient associated with the cornea, a quantitative parameter of the cornea.

2. The system of claim 1, wherein the OCT data is representative of an amplitude change of the reflected version of the optical signal.

3. The system of claim 1, wherein the scanning sequence comprises B-M Mode scanning wherein the OCT scanner is configured to:
perform a plurality of B-scan OCT images corresponding to two-dimensional, cross-sectional images of the ocular tissue;
perform a plurality of M-mode OCT images; and
determine an average of the plurality of B-scan OCT images.

4. The system or claim 3, wherein the performing the plurality of scans of the ocular tissue comprises:
perform a first scan in a location of the cornea corresponding to a first time;
perform a second scan in the location of the cornea corresponding to a second time;
determine a difference between a first measurement and a second measurement, based on the OCT data of the first scan or the second scan;
perform one or more subsequent scans in the location corresponding to one or more subsequent times; and
determine the optical signal based on an average value of the difference between the first measurement and the second measurement and one or more differences between the first measurement and one or more subsequent measurements.

5. The system of claim 4, wherein the determine the difference between the first measurement and the second measurement includes comparing an amplitude or a phase of the first measurement to the amplitude or phase of the second measurement.

6. The system of claim 1, wherein the determine the rate of change of the phase data corresponds to the plurality of scans.

7. The system of claim 1, wherein the performing a plurality of scans comprises B-M scan mode or M-B scan mode.

8. The system of claim 1, wherein the performing a plurality of scans comprises performing at least two scans simultaneously at least two different locations of the cornea.

9. The system of claim 8, wherein the performing a plurality of scans comprises performing three scans simultaneously on at least three different locations of the cornea.

10. A method of measuring ocular tissue characteristics of a cornea in real-time at multiple ocular locations, the method comprising:
at an optical coherence tomography (OCT) device:
performing a plurality of scans of the ocular tissue of the cornea to obtain OCT data representative of an OCT signal including phase data and amplitude data;
at a computer coupled to the OCT device:
determining a rate of change of the amplitude data or the phase data associated with the plurality of scans, the rate of change of the phase data or the amplitude data characterizing a change of a reflected version of the optical signal as a function of time;
determining a growth constant of the reflected version of the optical signal based on the rate of change of the amplitude data or the phase data;
determining, based on the growth constant, a diffusion coefficient associated with the ocular tissue; and
determining, based on the diffusion coefficient associated with the ocular tissue, a quantitative parameter of the ocular tissue.

11. The method of claim 10, wherein the plurality of scans comprise B-M Mode scanning, and wherein the OCT device is further configured to:
performing a plurality of B-scan OCT images corresponding to two-dimensional, cross-sectional images of the cornea;
performing a plurality of M-mode OCT images; and
determining an average of the plurality of B-scan OCT images.

12. The method of claim 10, wherein the performing the plurality of scans of the cornea comprises:
performing a first scan in a location of the cornea corresponding to a first time;
performing a second scan in the location of the cornea corresponding to a second time;
determining a difference between a first measurement and a second measurement of the first scan or the second scan;
performing one or more subsequent scans in the location corresponding to one or more subsequent times; and
determining the OCT signal based on an average value of the difference between the first measurement and the second measurement and one or more differences between the first measurement and one or more subsequent measurements.

13. The method of claim 12, wherein the determining the difference between the first measurement and the second measurement includes comparing an amplitude or a phase of the first measurement to the amplitude or phase of the second measurement.

14. The method of claim 10, wherein performing the plurality of scans comprises M-B scan mode.

15. The method of claim 10, wherein performing the plurality of scans comprises B-M scan mode.

16. The method of claim 10, wherein performing the plurality of scans comprises performing at least 2 scans simultaneously at least two different locations of the cornea.

17. The method of claim 10, wherein the performing a plurality of scans comprises performing three scans simultaneously on at least three different locations of the cornea.

18. A computer-program product comprising a non-transitory computer-usable medium having computer-readable program code embodied therein, the computer-readable program code adapted to be executed by one or more processors to implement a method comprising:
receiving, from an optical coherence tomography (OCT) device, OCT data representative of an optical signal including phase and amplitude information, the OCT data being generated by performing a plurality of scans of a cornea;
determining a rate of change of amplitude data or phase data associated with the plurality of scans, the rate of change of the phased data or amplitude data characterizing a change of a reflected version of the optical signal as a function of time;
determining a growth constant of the reflected version of the optical signal based on the rate of change of the amplitude data or the phase data;
determining, based on the growth constant, a diffusion coefficient associated with the cornea; and
determining, based on the diffusion coefficient associated with the cornea, a quantitative parameter of the cornea.

19. The computer-program product of claim 18, wherein:
the performing the plurality of scans of the cornea includes:
performing a first scan in a location of the cornea corresponding to a first time, and
performing a second scan in the location of the cornea corresponding to a second time; and
the method further includes:
determining a difference between a first measurement and a second measurement, based on OCT data of the first scan or the second scan;
providing, to the OCT device, control signals to perform one or more subsequent scans in the location corresponding to one or more subsequent times; and
determining the OCT signal based on an average value of the difference between the first measurement and the second measurement and one or more differences between the first measurement and one or more subsequent measurements.

20. The computer-program product of claim 19, wherein the determining the difference between the first measurement and the second measurement includes comparing an amplitude or a phase of the first measurement to the amplitude or phase of the second measurement.
